SEQUENCE LISTING (20 sequences)

SEQ ID NO: 1 - DNA, 20 bp, Mus musculus
  source 1..20: mol_type = other DNA
tgcagtggcaaagtggagat

SEQ ID NO: 2 - DNA, 20 bp, Mus musculus
  source 1..20: mol_type = other DNA
ggtctcgctcctggaagatg

SEQ ID NO: 3 - DNA, 20 bp, Mus musculus
  source 1..20: mol_type = other DNA
aatttgccaacgggcttgta

SEQ ID NO: 4 - DNA, 20 bp, Mus musculus
  source 1..20: mol_type = other DNA
ggcactggatcgtcctcttc

SEQ ID NO: 5 - DNA, 19 bp, Mus musculus
  source 1..19: mol_type = other DNA
ctgggagcacgctgcctat

SEQ ID NO: 6 - DNA, 21 bp, Mus musculus
  source 1..21: mol_type = other DNA
tttccgtatgttgtgctgcaa

SEQ ID NO: 7 - DNA, 21 bp, Mus musculus
  source 1..21: mol_type = other DNA
caagagccctgtctgtttcgt

SEQ ID NO: 8 - DNA, 20 bp, Mus musculus
  source 1..20: mol_type = other DNA
acaggagggcagagctcaca

SEQ ID NO: 9 - DNA, 20 bp, Mus musculus
  source 1..20: mol_type = other DNA
ctgggtgttgtggcggatat

SEQ ID NO: 10 - DNA, 20 bp, Mus musculus
  source 1..20: mol_type = other DNA
agcaaccaatggcccaatac

SEQ ID NO: 11 - DNA, 22 bp, Mus musculus
  source 1..22: mol_type = other DNA
ggctgtcgacctactggagaag

SEQ ID NO: 12 - DNA, 22 bp, Mus musculus
  source 1..22: mol_type = other DNA
ggctgtcgacctactggagaag

SEQ ID NO: 13 - DNA, 21 bp, Mus musculus
  source 1..21: mol_type = other DNA
tgactcaccttgtggtcctaa

SEQ ID NO: 14 - DNA, 22 bp, Mus musculus
  source 1..22: mol_type = other DNA
cttcccagaatccagtctttcc

SEQ ID NO: 15 - DNA, 20 bp, Mus musculus
  source 1..20: mol_type = other DNA
gtgtagttcccaagagcccc

SEQ ID NO: 16 - DNA, 17 bp, Mus musculus
  source 1..17: mol_type = other DNA
ccacagtttctcccaca

SEQ ID NO: 17 - DNA, 21 bp, Mus musculus
  source 1..21: mol_type = other DNA
tgccgacatgccaggacgaaa

SEQ ID NO: 18 - DNA, 20 bp, Mus musculus
  source 1..20: mol_type = other DNA
gtgggctctggtgggcgagt

SEQ ID NO: 19 - DNA, 20 bp, Mus musculus
  source 1..20: mol_type = other DNA
ccccttcaatggctggtaca

SEQ ID NO: 20 - DNA, 18 bp, Mus musculus
  source 1..18: mol_type = other DNA
gcgctggacgtcacagaa

(19) United States
(12) Patent Application Publication
(10) Pub. No.: US 2023/0242993 A1
(43) Pub. Date: Aug. 3, 2023

(54) METHOD FOR SCREENING MARKERS FOR PREDICTING PROGNOSIS OF RENAL CELL CARCINOMA USING TRANSCRIPTOMIC ANALYSIS

(71) Applicant: Gwangju Institute of Science and Technology, Gwangju (KR)

(72) Inventors: Hyun Ju LEE, Gwangju (KR); Darren Reece Williams, Gwangju (KR); Eui Young OH, Gwangju (KR); Da Woon JUNG, Gwangju (KR); Jun Hyeong KIM, Gwangju (KR)

(73) Assignee: Gwangju Institute of Science and Technology, Gwangju (KR)

(21) Appl. No.: 17/993,922

(22) Filed: Nov. 24, 2022

(30) Foreign Application Priority Data
Nov. 29, 2021 (KR) 10-2021-0166728

(51) Int. Cl. C12Q 1/6886; A61K 31/427; A61P 35/00

(52) U.S. Cl. CPC C12Q 1/6886; A61K 31/427; A61P 35/00; C12Q 2600/118; C12Q 2600/158

(57) ABSTRACT

A marker composition for predicting RCC prognosis, includes at least one gene selected from the group consisting of DUSP22, MAPK14, MAPKAPK3, STAT1, and VCP, discovered by the screening method of the present invention, or a protein encoded by the gene, and a pharmaceutical composition for treating RCC, includes a DUSP22 inhibitor of the present invention as an active ingredient.

### BACKGROUND OF THE INVENTION

### Cross-Reference to Related Applications

This application claims, under 35 U.S.C. § 119, the priority of Korean Patent Application No. 10-2021-0166728 filed on Nov. 29, 2021 in the Korean Intellectual Property Office, the disclosure of which is incorporated herein by reference in its entirety.

### SEQUENCE LISTING

This application contains a Sequence Listing submitted via EFS-Web and hereby incorporated by reference in its entirety. The Sequence Listing is named SEQCRF_2550-041.xml, created on Nov. 20, 2022, and 20,480 bytes in size.

### FIELD OF THE INVENTION

The present invention relates to a method for screening markers for predicting prognosis of renal cell carcinoma (RCC) using transcriptomic analysis, a marker for predicting the prognosis of RCC and a pharmaceutical composition for treating RCC, comprising a DUSP22 inhibitor as the active ingredients.

### DESCRIPTION OF THE RELATED ART

Renal cell carcinoma (RCC) is one of the malignant tumors occurring in the kidneys, accounting for 85% of cancers occurring in the kidneys. RCC is a metastatic disease with a very low 5-year survival rate of less than 10%, and it causes 15,000 deaths annually in the United States. Therefore, there is a need for developing markers capable of predicting the prognosis of RCC and for developing novel therapeutic targets and effective new therapeutic agents.

Cancer is considered as a degenerative disease related with aging, and the mechanism by which aging contributes to cancer progression is being studied actively. Numerous cellular phenomena related with aging, such as genomic instability, DNA damage, inflammation, and immune system disorders, are also known as features of cancer. Recently, the effects of the aging microenvironment on cancer have been extensively studied, providing new insights into cancer progression. The age of cells increases the secretion of aging-related cytokines, chemokines and growth factors, thereby causing tumor cell invasion. The integrity of extracellular matrix (ECM) decreases with aging, and changes in the ECM are related with tumor metastasis.

Although the aging microenvironment has been studied in cancer, few studies have investigated how changes in the expression of aging-related genes in normal tissues of cancer patients are related with genome-wide cancer progression. Several previous studies have characterized aging-related genes and their biological functions. For example, a previous study (Yang et al., 2015) was conducted in the Genotype-Tissue Expression (GTEx) project to analyze the potential association of synchronized changes of age-related gene expression across multiple tissues with degenerative diseases. In order to improve the understanding of aging and cancer, it is important to investigate the relationship between the expression level of aging-related genes in normal tissues and cancer progression or invasion.

Therefore, the inventors of the present invention investigated the relationship between changes of aging-related gene expression and genome-wide cancer progression through transcriptomic analysis, studied a marker screening method for predicting RCC prognosis, and suggested new therapeutic targets and therapeutic drugs.

### PRIOR ARTS

[Non-patent document] Cited Document D1: ang, J.; Huang, T.; Petralia, F.; Long, Q.; Zhang, B.; Argmann, C.; Zhao, Y.; Mobbs, C. V.; Schadt, E. E.; Zhu, J.; et al. Synchronized age-related gene expression changes across multiple tissues in human and the link to complex diseases. Sci. Rep. 2015, 5, 15145.

### SUMMARY OF THE INVENTION

The present invention provides a marker composition for RCC prognosis by screening a marker for predicting RCC prognosis using transcriptomic analysis, and a pharmaceutical composition for treating RCC, comprising a DUSP22 inhibitor.

To overcome the technical problem described above, according to one aspect of the present invention, is to provide the necessary information about RCC prognosis, the present invention provides a method for screening markers for predicting the prognosis of RCC using transcriptomic analysis, comprising:
a) identifying a group of aging-related genes by integrating the gene expression data and protein-protein interaction data of a normal tissue of RCC patients;
b) analyzing relationship between the expression of aging-related genes in the normal tissue and survival of cancer patients; and
c) determining an aging-related gene that is up-regulated in the normal tissue and shows a significant relationship with survival of RCC patients as a marker for predicting RCC prognosis.

According to one embodiment of the present invention, the a) may be performed by using a linear regression model.

According to one embodiment of the present invention, the b) may comprise validating the effect of aging-related gene expression on cancer cell invasion and metastasis through in vitro and in vivo experiments using aging animal models.

According to one embodiment of the present invention, the aging-related gene determined as a marker for predicting RCC prognosis in c) may be at least one gene selected from the group consisting of DUSP22, MAPK14, MAPKAPK3, STAT1, and VCP.

According to one embodiment of the present invention, the RCC may be clear cell renal cell cancer.

According to another aspect of the present invention, provided is a marker composition for predicting RCC prognosis, comprising at least one below (a) at least one gene selected from the group consisting of DUSP22, MAPK14, MAPKAPK3, STAT1, and VCP; and (b) protein encoded by at least one gene selected from the group consisting of DUSP22, MAPK14, MAPKAPK3, STAT1, and VCP.

According to another aspect of the present invention, provided is a pharmaceutical composition for treating RCC, comprising a DUSP22 inhibitor as an active ingredient.

According to one embodiment of the present invention, the DUSP22 inhibitor may be a compound represented by Formula 1 below.

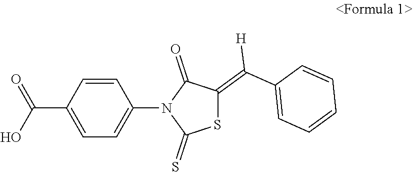 [SMILES: O=C(O)c1ccc(N2C(=O)/C(=C/c3ccccc3)SC2=S)cc1]

According to one embodiment of the present invention, the DUSP22 inhibitor may be a compound represented by the Formula below.

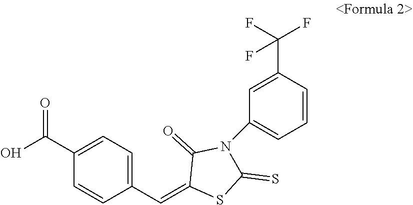 [SMILES: O=C(O)c1ccc(/C=C2/SC(=S)N(c3cccc(C(F)(F)F)c3)C2=O)cc1]

Through the marker composition for predicting RCC prognosis, comprising at least one gene selected from the group consisting of DUSP22, MAPK14, MAPKAPK3, STAT1, and VCP, discovered by the screening method of the present invention, or a protein encoded by the gene, the prognosis of RCC may be predicted.

In addition, through the pharmaceutical composition for treating RCC, comprising a DUSP22 inhibitor of the present invention as an active ingredient, RCC may be treated.

The effects of the present invention are not limited to the above-described effects, and should be understood as including all effects that can be inferred from the configuration of the invention described in the description or claims of the present invention.

### BRIEF DESCRIPTION OF THE DRAWINGS

The accompanying drawings are intended to explain the contents of the present invention in more details to those skilled in the art, but the technical principle of the present invention is not limited thereto.

FIG. 1 illustrates the entire flow of the screening method.

FIG. 2A illustrates a graph representing the Kaplan-Meier survival curve depending on the mean expression value of up-regulated aging genes.

FIG. 2B illustrates a graph representing the Kaplan-Meier survival curve depending on the mean expression value of down-regulated aging genes.

FIG. 2C illustrates a graph Kaplan-Meier survival curve depending on the mean expression value of five up-regulated aging genes (DUSP22, MAPK14, MAPKAPK3, STAT1 and VCP).

FIG. 2D illustrates a graph comparing the AUCs of survival prediction models having five different input variables.

FIG. 3 is a schematic of a protocol for evaluating the aging-related genes of monocyte-macrophage cells of young mice or old mice.

FIG. 4 is an image of immunofluorescent staining CD68 and cytokeratin in bone marrow-derived macrophages differentiated by RENCA cell conditioned medium (scale bar=100 μm).

FIG. 5A illustrates a graph representing qPCR analysis of the expression of MAPKAPK3 genes in bone marrow-derived monocytes isolated from young mice (5 weeks old) or old mice (72 weeks old).

FIG. 5B illustrates a graph representing qPCR analysis of the expression of MAPK14 genes in bone marrow-derived monocytes isolated from young mice (5 weeks old) or old mice (72 weeks old).

FIG. 5C illustrates a graph representing qPCR analysis of the expression of DUSP22 genes in bone marrow-derived monocytes isolated from young mice (5 weeks old) or old mice (72 weeks old).

FIG. 5D illustrates a graph representing qPCR analysis of the expression of STAT1 genes in bone marrow-derived monocytes isolated from young mice (5 weeks old) or old mice (72 weeks old).

FIG. 5E illustrates a graph representing qPCR analysis of the expression of VCP genes in bone marrow-derived monocytes isolated from young mice (5 weeks old) or old mice (72 weeks old).

FIG. 6 is a representative image of invaded RENCA renal adenocarcinoma cells co-cultured with bone marrow-derived macrophages isolated from young mice or old mice (scale bar=100 μm).

FIG. 7 is a graph quantifying invaded RENCA renal adenocarcinoma cells co-cultured with bone marrow-derived macrophages isolated from young mice or old mice.

FIG. 8A is a graph representing qPCR analysis of DUSP22 gene expression in RAW264.7 cells of scrambled control or the cells after transfection with DUSP22 or MAPK14 siRNA.

FIG. 8B is a graph representing qPCR analysis of MAPK14 gene expression in RAW264.7 cells of scrambled control or the cells after transfection with DUSP22 or MAPK14 siRNA.

FIG. 9 is a representative image of invaded RENCA RCC cells co-cultured with RAW264.7 cells of scrambled control or the cells after transfection with DUSP22 or MAPK14 siRNA.

FIG. 10 is a graph quantifying invaded RENCA RCC cells co-cultured with RAW264.7 cells of scrambled control or the cells after transfection with DUSP22 or MAPK14 siRNA.

FIG. 11 is an image representing the inhibitory effect of DUSP22 target drug BML-260 on invasion analysis of RENCA RCC cells co-cultured with RAW264.7 macrophages (scale bar=100 μm).

FIG. 12 is a graph quantifying the inhibitory effect of DUSP22 target drug BML-260 on the invasion analysis of RENCA RCC cells co-cultured with RAW264.7 macrophages.

FIG. 13 is a schematic of the protocol for investigating the macrophage DUSP22 inhibitory effect on dissemination (early stage of metastasis) in co-transplanted RCC cells in vivo.

FIG. 14 is a representative image of zebrafish larvae after single transplantation of RCC cells (n=39), or co-transplantation with DMSO-treated macrophages (n=39), or co-transplantation with BML-260-treated macrophages (n=44) (scale bar=100 μm).

FIG. 15 is a graph representing the RCC dissemination rate of zebrafish after single transplantation of RCC cells (n=39), co-transplantation with DMSO-treated macrophages (n=39), or co-transplantation with BML-260-treated macrophages (n=44)

FIG. 16 is an image representing the change of the invasion of RCC cells depending on CFTRinh-172 treatment of macrophages.

FIG. 17 is a graph quantifying the change of the invasion of RCC cells depending on CFTRinh-172 treatment of macrophages.

### DETAILED DESCRIPTION OF THE EMBODIMENTS

Hereinafter, the present invention will be described in more detail. However, this is presented as an example, and the present invention is not limited thereto, and the present invention is only defined by the scope of the claims to be described later.

To provide the necessary information about RCC prognosis, the present invention provides a method for screening markers for predicting prognosis of RCC using transcriptomic analysis, comprising:
a) identifying a group of aging-related genes by integrating the gene expression data and protein-protein interaction data of a normal tissue of RCC patients;
b) analyzing relationship between the expression of aging-related genes in the normal tissue and survival of cancer patients; and
c) determining an aging-related gene that is up-regulated in the normal tissue and shows a significant relationship with the survival of RCC patients as a marker for predicting RCC prognosis.

In the present invention, the a) may be performed by using a linear regression model.

In the present invention, the b) may comprise validating the effect of aging-related gene expression on cancer cell invasion and metastasis through in vitro and in vivo experiments using animal models of aging.

In the present invention, the aging-related gene determined as a marker for predicting RCC prognosis in c) may be at least one gene selected from the group consisting of DUSP22, MAPK14, MAPKAPK3, STAT1, and VCP.

In the present invention, the RCC may be clear cell renal cell cancer, but is not limited thereto. The clear cell renal cell cancer, the ninth most common cancer, is reported as a small renal tumor with a diameter of less than 40 mm. Clear cell renal cell cancer is the most common type of renal cancer, accounting for about 80% of renal cancers, and is generated from the renal proximal tubule. Clear cell renal cell cancer is characterized by abundant blood vessels, easy metastasis to other organs, and response to targeted therapy and immunotherapy.

The present invention provides a marker composition for predicting RCC prognosis, comprising at least one below (a) at least one gene selected from the group consisting of DUSP22, MAPK14, MAPKAPK3, STAT1, and VCP; and (b) protein encoded by at least one gene selected from the group consisting of DUSP22, MAPK14, MAPKAPK3, STAT1, and VCP.

The present invention provides a pharmaceutical composition for treating RCC, comprising a DUSP22 inhibitor as an active ingredient.

In the present invention, the DUSP22 inhibitor may be a compound represented by Formula 1 below. The compound represented by Formula 1 below is BML-260, which is known as a DUSP22 inhibitor.

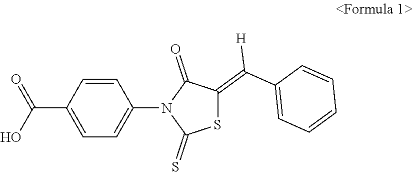 [SMILES: O=C(O)c1ccc(N2C(=O)/C(=C/c3ccccc3)SC2=S)cc1]

In the present invention, the DUSP22 inhibitor may be a compound represented by Formula below. The compound represented by Formula 2 below is CFTRinh-172, which is a derivative of BML-260.

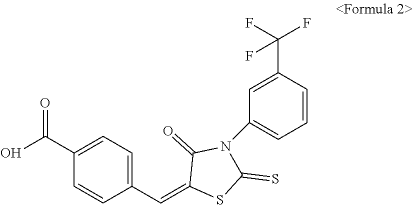 [SMILES: O=C(O)c1ccc(/C=C2/SC(=S)N(c3cccc(C(F)(F)F)c3)C2=O)cc1]

Hereinafter, the present invention will be described in more details through Examples and Experimental Examples. However, the following Examples and Experimental Examples are intended to illustrate the present invention, and the scope of the present invention is not limited thereto.

### EXAMPLES

### 1. Materials and Methods

### 1.1 Overview of Screening Method

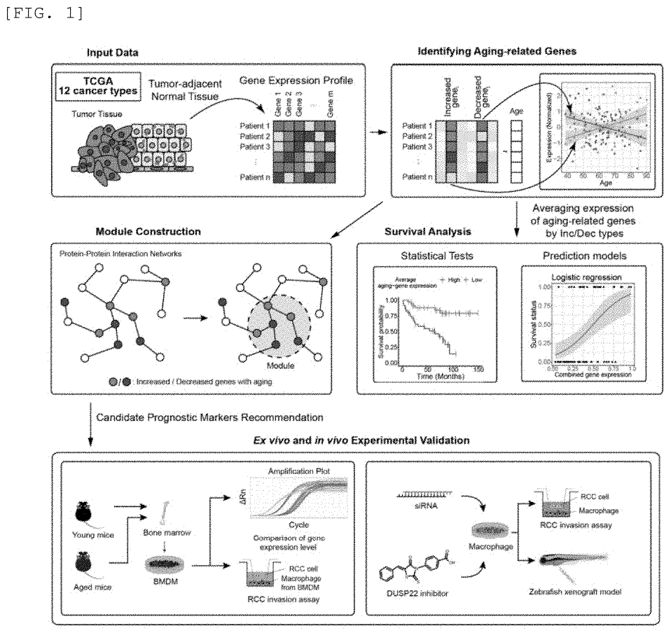

FIG. 1 illustrates the entire flow of the screening method. Gene expression data sets of normal and tumor cells from cancer patients were collected from The Cancer Genome Atlas (TCCA). The types and abbreviations of the selected cancers are bladder cancer (BLCA), breast cancer (BRCA), kidney-related cancer (KICH, KIRC, KIRP), lung cancer (LUAD, LUSC), head and neck cancer (HNSC), liver cancer (LIHC), stomach cancer (STAD), thyroid cancer (THCA), and uterine cancer (UCEC). Through linear regression and DESeq2 (RRID:SCR_000154), normal transcriptome and tumor transcriptome data sets were each compared to identify aging-related genes and microRNAs (miRNAs). To find representative aging-related clusters, a dynamic tree cut method was used to map the aging-related genes and constructed modules on the protein-protein interaction network. DAVID (RRID:SCR_001881) and logistic regression models were used to each investigate the enhanced biological function and survival predictive performance of the identified aging-related genes. Through the Cox regression and Kaplan-Meier estimator, the relationship between the expression level of the identified genes and the patient's survival was investigated. Aged animal models were used to validate the effect of the expression of the identified aging-related genes on cancer cell invasion and metastasis.

### 1.2 Materials

The inventors of the present invention compared mRNA and miRNA expression profiles between normal and tumor tissues derived from data set types BLCA, BRCA, KICH, KIRC, KIRP, LUAD, LUSC, HNSC, LIHC, STAT, THCA and UCCC of the same patients.

Expression of mRNA and miRNA was measured by using Illumina HiSeq-RNASeqV2 and Illumina HiSeq-miRNASeq, respectively. Fragments Per Kilobase of transcript per Million mapped reads (FPKM-UQ) were used to identify aging-related genes each in normal tissues and DEGs of tumor tissues. The results of the miRNA expression were recorded as number of reads and Reads Per Million (RPM). The data was downloaded from the TCGA data portal by using TCGA Biolink.

### 1.3 Data Preprocessing

TCGA transcript profiling data includes protein-coding genes and non-coding regions such as pseudogenes and noncoding RNAs. After filtering the non-coding regions based on Ensembl (RRID:SCR_002344) gene biotype, 19,589 protein-coding genes were obtained. Then, to reduce the number of unreliable results in each tissue type, genes having a non-zero expression value in more than 30 samples were selected. When the number of samples was less than 30, genes having an expression value of 0 in more than 30% of the samples were removed. Since miRNA expression values are expressed as RPM values, quantile normalization for miRNA expression data was used to enable comparison between samples. In addition, when more than 70% of the samples had a miRNA expression value of 0, the miRNAs were filtered out.

1.4 Identification of Aging-Related Genes and miRNAs

The method for identifying aging-related genes and miRNAs described below is based on previous studies conducted by using a linear model. Since again is a continuous process, not a discrete event, a linear regression model was used to identify aging-related genes and miRNAs.

$$M 1 : {Expression}_{ij} = {β}_{j} + {γ}_{j} {Age}_{i} + {e}_{ij} , \qquad ( 1 )$$

$$M 2 : {Expression}_{ij} = {β}_{j} + {γ}_{j} {Age}_{i} + {\sum }_{k = 1}^{N} α {j}_{k} {PCi}_{k} + {e}_{ij} , \qquad ( 2 )$$

Here, Expressionᵢⱼ represents the expression value of the gene or miRNA j in sample i. Ageᵢ represents the age at initial diagnosis in sample i, βⱼ the regression intercept of gene j, γⱼ the regression coefficient of age, and εᵢⱼ the error term. PCᵢₖ represents the value of the kth principal component of the gene expression data for ith sample. Of the top 10 PCs, N PCs having no significant correlation with age ((p-value)>0.05 for Pearson's correlation test) were selected to increase the significance level of the age coefficient.

The inventors of the present invention selected genes and miRNAs having an age regression coefficient corresponding to a p-value less than 0.05 in the two linear regression models. Age-related genes and miRNAs were classified into increasing genes and decreasing genes according to the signs of the age coefficient of the linear regression models. To confirm whether the detected aging-related genes are sensitive to a particular configuration, the data was resampled by bootstrapping.

After identifying the aging-related genes, the mean expression values of increasing genes and decreasing genes were calculated, which were referred to as “increasing index” and “decreasing index,” respectively.

To investigate whether the expression values of the confirmed aging-related genes have a significant effect on the survival time of cancer patients, Cox's proportional hazard model and Kaplan-Meier estimator were used.

### 1.5 Finding Differentially Expressed Genes (DEG) in Cancer

To find DEGs in cancer, DESeq2 (RRID:SCR_000154) was used. Expression data of tumor tissues and normal tissues from the same patients were paired. The inventors of the present invention constructed a DESeq2 model with gene expression as a dependent variable and with tissue types and patient IDs as independent variables. When log 2-fold-change between normal and cancer tissues was greater than 2 and the adjusted p-value determined by the Benjamini-Hochberg method was less than 0.01, the gene was considered as having been differentially expressed.

### 1.6 Construction of Modules by Using Protein-Protein Interaction Network

To construct a module comprising functionally associated aging-related genes, a protein-protein interaction database provided by Human Protein Reference Database (HPRD) was used. The protein-protein interaction database from the HPRD was compiled, a network was configured, and aging-related genes were mapped. The similarity distance between two aging-related genes in the network was calculated by using the diffusion kernel method. A hierarchical cluster tree was constructed based on the differences in aging-related genes, and modules were defined by using a dynamic tree cutting method.

### 1.7 Experimental Validation

### 1.7.1 Reagents for Experiment

Collagen type I was purchased from Roche (Basel, Switzerland). Lipofectamine 3000 transfection kit (L300008), Silencer negative control siRNA (AM4611), mouse Dusp22 siRNA (#287290) and Mapk14 siRNA (#240556) were purchased from Invitrogen (Waltham, Mass., USA). Anti-CD68 (ab201340) and anti-cytokeratin (ab9377) antibodies were purchased from Abcam (Cambridge, UK). BML-260 (sc-223822) was purchased from Santa Cruz Biotechnology (Dallas, Tex., USA). CFTRinh-172 (C2992) was purchased from Sigma-Aldrich (Burlington, Mass., USA).

### 1.7.2 Cell Culture

RENCA mouse renal epithelial adenocarcinoma cells and RAW264.7 mouse macrophages were purchased from the Korean Cell Line Bank (Seoul, Korea). RENCA and RAW264.7 cells were maintained in Dulbecco's Modified Eagle Medium (DMEM) medium (Gibco, Thermo Fisher Scientific, Waltham, Mass., USA) supplemented with 10% FBS and 1% penicillin/streptomycin.

### 1.7.3 Collection of Conditioned Medium

To collect conditioned medium (CM), cells were cultured to 70% to 80% confluency in 10 cm culture dishes, and the medium was replaced with serum-free medium. Then, 48 hours later, the CM was collected and centrifuged at 1500 rpm for 3 minutes at 4° C. The CM was then filtered by using a 0.2 μm syringe filter and stored at −20° C. until use.

### 1.7.4 SiRNA-Mediated Gene Knockdown

Cells were seeded in a 6-well plate for RNA isolation and in a 24-well plate for invasion analysis. After incubation for 24 hours, Lipofectamine 3000 was used to prepare a transfection medium.

Lipofectamine 3000 and siRNA were diluted in DMEM, mixed at a ratio of 1:1, and the resulting mixture was incubated for 15 minutes at room temperature (RT). Then, the cells were washed with PBS and treated with an appropriate volume of transfection medium. Experiments were performed in 24 hours after the transfection.

### 1.7.5 Real-Time Quantitative PCR

Each cDNA was synthesized from RNA by using AccuPower RT PreMix (Bioneer, Daejeon, Korea) according to the manufacturer's protocol. Each sample was tested in triplicate by qPCR in a total volume of 20 μL, containing 10 μL of ToPreal qPCR 2×PreMIX (Enzynomics, Daejeon, Korea), 250 nM of specific forward and reverse primers, and 1 μL of cDNA. The initial denaturation step was performed at 95° C. for 10 min, and the amplification step consisted of 40 cycles of denaturation, annealing and extension. Denaturation was performed at 95° C. for 15 seconds, and annealing and expansion were performed at 60° C. for 1 minute. After the last cycle, the melting points of all samples were analyzed within the range of 60-95° C. through continuous fluorescence detection. Gene expression was normalized to the expression of GAPDH. Table 1 shows the details of the primers.

TABLE 1
qPCR primer list

| | | |
|---|---|---|
| Mouse GAPDH | Forward | TGCAGTGGCAAAGTGGAGAT (SEQ ID NO: 1) |
| | reverse | GGTCTCGCTCCTGGAAGATG (SEQ ID NO: 2) |
| Mouse VCP | Forward | AATTTGCCAACGGGCTTGTA (SEQ ID NO: 3) |
| | reverse | GGCACTGGATCGTCCTCTTC (SEQ ID NO: 4) |
| Mouse STAT1 | Forward | CTGGGAGCACGCTGCCTAT (SEQ ID NO: 5) |
| | reverse | TTTCCGTATGTTGTGCTGCAA (SEQ ID NO: 6) |
| Mouse DUSP22 | Forward | CAAGAGCCCTGTCTGTTTCGT (SEQ ID NO: 7) |
| | reverse | ACAGGAGGGCAGAGCTCACA (SEQ ID NO: 8) |
| Mouse MAPKAPK3 | Forward | CTGGGTGTTGTGGCGGATAT (SEQ ID NO: 9) |
| | reverse | AGCAACCAATGGCCCAATAC (SEQ ID NO: 10) |
| Mouse DUSP22 | Forward | GGCTGTCGACCTACTGGAGAAG (SEQ ID NO: 11) |
| | reverse | AGGGTCGTGGTACTGAGCAAA (SEQ ID NO: 12) |
| Mouse F4/80 | Forward | TGACTCACCTTGTGGTCCTAA (SEQ ID NO: 13) |
| | reverse | CTTCCCAGAATCCAGTCTTTCC (SEQ ID NO: 14) |
| Mouse CD68 | Forward | GTGTAGTTCCCAAGAGCCCC (SEQ ID NO: 15) |
| | reverse | CCACAGTTTCTCCCACA (SEQ ID NO: 16) |
| Mouse CD206 | Forward | TGCCGACATGCCAGGACGAAA (SEQ ID NO: 17) |
| | reverse | GTGGGCTCTGGTGGGCGAGT (SEQ ID NO: 18) |
| Mouse iNOS | Forward | CCCCTTCAATGGCTGGTACA (SEQ ID NO: 19) |
| | reverse | GCGCTGGACGTCACAGAA (SEQ ID NO: 20) |

### 1.7.6 Invasion Analysis

Cancer cell invasion analysis was performed in a 24-well transwell plate (Corning Costar, New York, N.Y., USA). A transwell having a pore size of 8 μm was coated with Type I collagen (3 μg/60 μL/well). A total of 4×10⁴ bone marrow-derived macrophage (BMDM) cells were seeded in the lower chamber and differentiated into macrophages by using cancer CM. A total of 1×10⁴ cancer cells were seeded into the upper transwell. After 24 or 48 hours, cancer cells that invaded into the lower chamber through the porous membrane were fixed with 3.7% formaldehyde and stained with 0.2% crystal violet. Images of the stained cells were obtained by using an optical microscope (CKX41, Olympus, Tokyo, Japan) and analyzed by using the ImageJ software program (NIH, Bethesda, Md., RRID:SCR_003070). To evaluate the effect of DUSP22 or MAPK14 knockdown, 1×10⁵ RAW264.7 cells were seeded in the lower chamber and transfected with siRNA.

### 1.7.7 Immunocytochemistry

Cancer CM was used to differentiate BMDMs in a 24-well culture plate for 3 days. Cells were immunostained for the pan-macrophage marker CD68 or the epithelial cell marker cytokeratin. Cells were fixed by using a 3.7% paraformaldehyde solution for 10 min at room temperature.

Cells were washed with a PBST solution (1×PBS containing 0.1% Tween-20) and then permeabilized with 0.25% Triton X-100 for 10 min at room temperature. After washing, non-specific binding of an antibody to the cells was blocked with 1% BSA, 22.52 mg/mL glycine for 30 min at room temperature, and the resulting cells were incubated overnight at 4° C. with CD68 or cytokeratin antibody.

Alexa Fluor 488 goat anti-mouse IgG or Alexa Fluor 594 goat anti-rabbit IgG was used as a secondary antibody (Abcam, Cambridge, UK). A secondary antibody in 1% BSA was applied to the cells for 1 hour at room temperature. Nuclei were stained by using DAPI. Staining was visualized with a fluorescence microscope (Leica DMI3000 B).

### 1.7.8. Enzyme-Linked Immunosorbent Assay (ELISA)

ELISA on the CM was performed by using a commercial kit according to the manufacturer's protocol (M-CSF and GM-CSF kits were purchased from R&D Systems). To quantify the protein level of M-CSF or GM-CSF, the CM was harvested after 48 hours of incubation in a serum-free medium.

### 1.7.9 Animals

Animal experiments were approved by the Animal Management and Use Committee (GIST-2019-042) of the Gwangju Institute of Science and Technology. Mice were provided by Damul Science (Daejeon, Korea) and Orient Bio (Gyeonggi, Korea). To perform in vitro experiments, BMDMs were isolated from the femurs and tibias of C57BL/6 male mice.

BMDMs were collected from the bones by using a 30 G syringe and ice-cold sterile PBS. Then, the cells were filtered through a 70 μm cell strainer to remove tissue debris and centrifuged at 300×g for 10 min at 4° C. An erythrocyte lysis buffer of 5 mL was added for 5 min, and 5 mL PBS was added, and then the sample was centrifuged to remove erythrocytes from the pellet.

The BMDM cells were cultured in a 6-well ultra-low attachment plate in a monocyte medium (10% fetal bovine serum, 1% penicillin/streptomycin, 1× Glutamax (Gibco, Thermo Fisher Scientific), 1 mM sodium pyruvate, 1× non-essential amino acids, 10 mM HEPES, 55 μM β-mercaptoethanol and 10 ng/mL M-CSF (Peprotech, Rocky Hill, N.J., USA)) and were maintained at 37° C. for 5 days. To remove non-monocytes from the cell population, monocytes were purified by negative selection performed by using anti-CD117 magnetic microbeads (Miltenyi Biotec, Bergisch Gladbach, Germany) with a magnetic separator.

### 1.7.10. Zebrafish-Human Cancer Xenotransplantation Model

Evaluation of in vivo mouse RCC cell dissemination (early stage of metastasis) was performed by using a validated zebrafish model.

Embryos of 48 hours post-fertilization were prepared for cell xenotransplantation and either 39 or 44 embryos were used for each treatment group. After the RCC cells were stained with 2 μg/mL DiI (Invitrogen), the villus of the embryos was removed, and then the embryos were anesthetized with 0.0016% tricaine. An injector (Picospritzer [symbol] , Parker Hannifin, Cleveland, Ohio, USA) was used to inject 200 RCC cells into the center of egg yolk or a mixture of the RCC cells and DMSO-treated macrophages or of the RCC cells and DUSP inhibitor-treated macrophages (1:1 ratio) was injected to the center of egg yolk. The xenotransplanted embryos were transferred to a 96-well plate of 200 μL of E3 medium. The number of embryos, representing the dissemination of cancer cells from the injection site, was counted by using a fluorescence microscope (Leica DM2500 Microscope).

### 1.7.11 Statistics for Cell-Based Analysis

A parametric Student's t-test was used for statistical analysis. A p-value less than 0.05 was considered significant.

### 2. Results

### 2.1 Data Characteristics

### 2.1.1 Demographics

Table 2 shows the number of samples, demographic characteristics, and patient survival rates for specific cancer types. The age of the patients ranged from 20 to 90 years with the highest number of patients at 60 years. The mean and standard deviation by age were 61±14.8.

TABLE 2

| | Sample Size | | | | | | | | | | |
|---|---|---|---|---|---|---|---|---|---|---|---|
| Cancer | (Survival/Deceased) | | | | Mean | Aging Genes | | | Aging microRNAs | | |
| Type | Gene | | microRNA | | Age | Increasing | Decreasing | Total | Increasing | Decreasing | Total |
| BLCA | 19 | (8/1 [symbol] ) | 19 | (8/11) | 70.32 | 43 | 94 | 137 | 2 | 3 | 5 |
| BRCA | 113 | (69/44) | 1 [symbol] 4 | (61/4 [symbol] ) | [symbol] 7.98 | 772 | 1 [symbol] 7 [symbol] | 2450 | 45 | 42 | 87 |
| HNSC | 44 | (11/33) | [symbol] 4 | (11/33) | 62.6 [symbol] | 52 | 62 | 1 [symbol] | 5 | 9 | 14 |
| KICB | 24 | (20/4) | 25 | (21/4) | 54.5 [symbol] | 202 | 139 | 341 | 5 | 10 | 15 |
| KIRC | 72 | (45/27) | 71 | (45/26) | 62.9 [symbol] | 162 | 87 | 249 | 17 | 15 | 32 |
| KI [symbol] | 32 | (2 [symbol] /7) | 3 [symbol] | (2 [symbol] / [symbol] ) | 62.4 [symbol] | 137 | 215 | 352 | 9 | 20 | 29 |
| LIHC | 50 | ( [symbol] 6/34) | 50 | (16/34) | [symbol] | 13 | 6 [symbol] | 78 | 8 | 18 | 26 |
| LOAD | 59 | (33/26) | 46 | (33/13) | 65.8 [symbol] | 339 | 289 | 628 | 12 | 11 | 23 |
| LOSC | 49 | (29/20) | 45 | (22/23) | 69.25 | 21 | 36 | [symbol] 7 | 8 | 13 | 21 |
| STAD | 32 | (23/9) | 45 | (33/12) | 69.25 | 14 | 0 | 14 | 4 | 3 | 7 |
| THCA | 58 | ( [symbol] 4/4) | 89 | ( [symbol] 5/4) | 46.0 [symbol] | 385 | 20 [symbol] | 591 | 34 | 36 | 7 [symbol] |

| | | | | | | | | | |
|---|---|---|---|---|---|---|---|---|---|
| UCBC | 35 (20/3, 12 | 3 [symbol] (19/3, 11 | 59.87 | 15 | 6 | 21 | 12 | 16 | 28 |
| | Not Available) | Not Available) | | | | | | | |

[symbol]

indicates data missing or illegible when filed

### 2.1.2 Chronological Survival Analysis

To find out whether patient age and survival are related, a univariate Cox regression model that predicts survival by using age as an input variable for each cancer type was constructed. The results showed that age was a significant risk factor for KIRC and THCA, and their p-values were 0.002 and 0.026, respectively. On the contrary, age was not related with survival in other cancer types, because the p-value was not significant according to the univariate Cox regression model.

2.2 Tissue-Specific Aging-Related Gene and miRNA

Table 2 shows the number of aging-related genes and miRNAs identified for each cancer type. Only the expression of 20 genes (12 up-regulated and 8 down-regulated genes) and 6 miRNAs (2 up-regulated and 4 down-regulated miRNAs) out of the aging-related genes and miRNAs were significantly different in 3 or more tissue types, suggesting that the change of the expression levels in aging is tissue-specific. In particular, ZNF518B was identified in six tissue types, including BRCA, KICH, LIHC, LUSC, KIRP and THCA, and NEFH was detected in four tissue types, including BRCA, KIRC, LUAD and THCA.

A pathway enrichment test was performed for each tissue and for each increase/decrease type of the aging-related genes. The inventors of the present invention applied the Database for Annotation, Visualization and Integrated Discovery (DAVID) v6.7 tool for GO (Gene Ontology) and KEGG (Kyoto Encyclopedia of Genes and Genomes).

The results particularly showed that pathways related with immune system, cell cycle, and metabolic processes are often rich in aging-related genes. This biological process is also known to be related with cancer occurrence, showing a relationship between the expression of aging-related genes and cancer occurrence. However, the results showed that altered aging-related pathways generally differ between tissue types.

### 2.3. Association Between Survival and Aging-Related Genes in BLCA, BRCA and THCA

The relationship between aging-related genes and patient survival rates was investigated for each tissue and for each increase/decrease type. In the case of BLCA, BRCA and THCA, expression levels of decreasing aging-related genes were significantly related with patient survival.

### 2.4 KIRC Analysis

### 2.4.1 Association Between Survival and Aging-Related Genes in TCGA-KIRC

The mean expression of all aging-related transcripts in KIRC was related with survival. In the univariate Cox model, the increase indices derived from 162 up-regulated genes had a hazard ratio of 2.27 and a p-value of 2.09×10⁻⁵, whereas the decrease indices derived from 87 down-regulated genes had a hazard ratio of 0.48 and a p-value of 0.01.

When patients were divided into two groups based on the median of the increase or decrease indices, the results of the Kaplan-Meier estimator were consistent with those of the Cox model. The Kaplan-Meier model showed that when the criterion was an increase or decreasing index, the p-values in the log-rank test were 6.78×10⁻⁵ or 8.52×10⁻⁴ respectively.

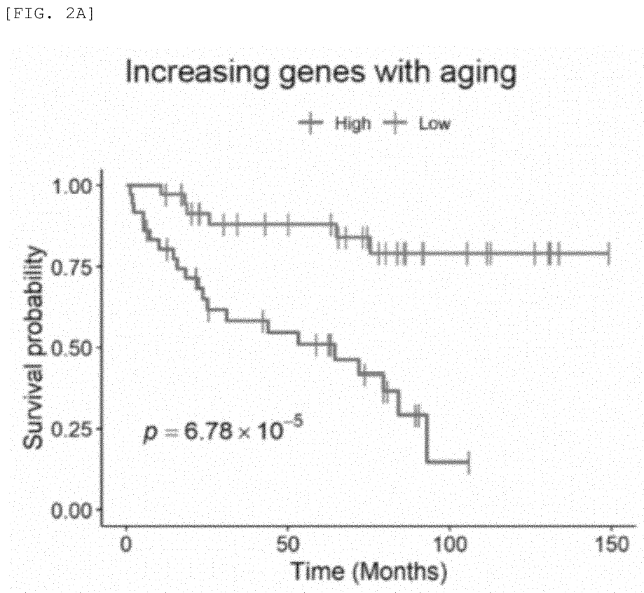

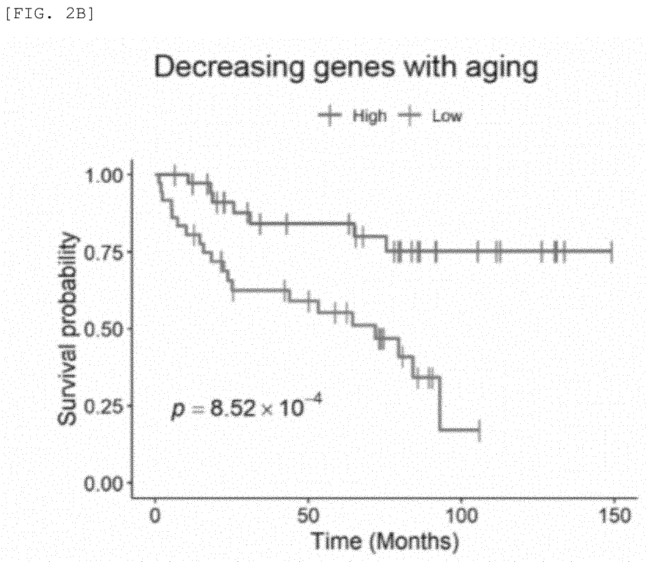

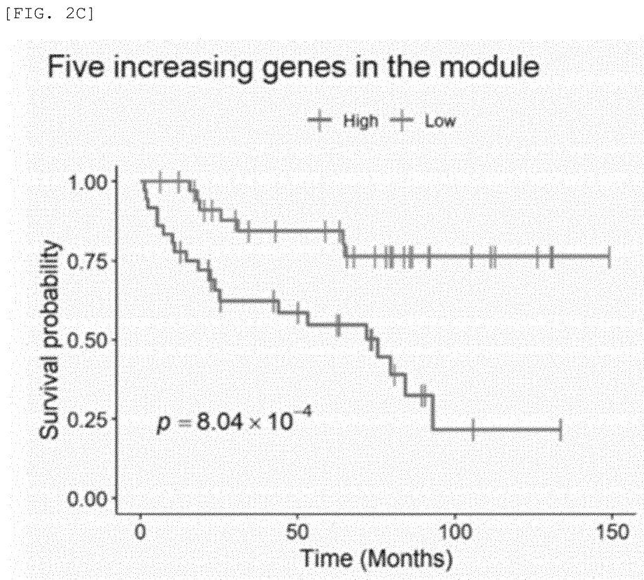

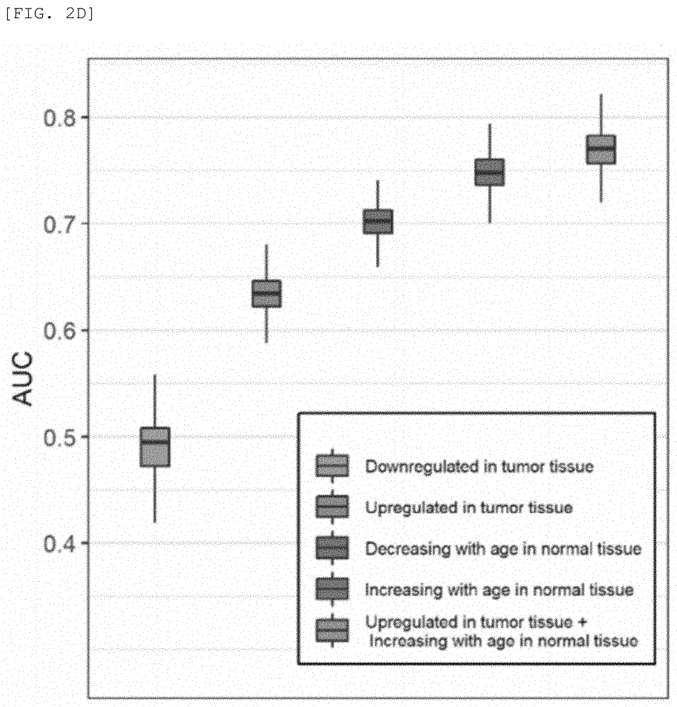

FIG. 2 shows the Kaplan-Meier overall survival curves of aging-related genes in TCGA-KIRC patients. These results showed that kidney cancer patients having a younger gene expression pattern in normal cells are more likely to live longer.

The graphs in FIG. 2 represent the survival analysis and prognostic ability of aging-related genes in KIRC. Patients were grouped according to the mean expression of up-regulated and down-regulated genes in TCGA-KIRC (FIG. 2A and FIG. 2B). The mean expression values of the five up-regulated genes (DUSP22, MAPK14, MAPKAPK3, STAT1 and VCP) in the module also had predictive power (FIG. 2C).

In addition, the AUC of the survival prediction model with 5 different input variables was compared (FIG. 2D). The expression level of aging-related genes in normal kidney tissue showed better survival predictive performance than DEG in KIRC tissue. The best performance was obtained when both the aging-related gene and DEG were used at the same time.

Of the aging-related miRNAs identified in KIRC, 17 miRNAs were up-regulated and 15 were down-regulated. In the Cox model, the increase indices of the 17 up-regulated miRNAs had a hazard ratio of 1.67 and a p-value of 0.008, and the decrease indices of the 15 down-regulated miRNAs had a hazard ratio of 0.44 and a p-value of 7.89×10⁻⁴.

When the increase indices and decrease indices were used as independent variables, the p-values of the Kaplan-Meier model were 0.005 and 0.03, respectively. These results were consistent with the results showing that age is a significant risk factor in KIRC. In particular, similar to aging-related genes, KIRC miRNAs were associated with survival. Based on these results, further analysis was conducted focusing on KIRC.

### 2.4.2 Module Analysis

To find a representative cluster of aging-related genes, the Human Protein Reference Database (HPRD) was used to construct a protein-protein interaction network comprising 73 up-regulated aging-related genes and 29 down-regulated aging-related genes of KIRC. Five up-regulated (DUSP22, MAPK14, MAPKAPK3, STAT1 and VCP) genes and three down-regulated (BRCA1, BRIP1 and NUFIP1) genes were detected based on the similarity between the mapped aging-related genes. These genes functioned as DNA damage checkpoints (Benjamini adjusted p-value 0.02), intracellular signaling mediators (0.02) and cell cycle checkpoints (0.03). Alterations of these biological pathways were observed in renal cancer patients.

Interestingly, the mean expression of the five up-regulated genes of KIRC was also associated with survival. The increase indices derived from DUSP22, MAPK14, MAPKAPK3, STAT1 and VCP in the Cox model have a hazard ratio of 1.86 and a p-value of 5.37×10⁻⁵. At the same time, the Kaplan-Meier estimator showed a p-value of 8.04×10⁻⁴ as shown in FIG. 2C. Indeed, the expression values of each of DUSP22, MAPKAPK3, VCP, and STAT1 had a significant relationship with survival (Cox-regression p-values of 0.01, 0.04, 0.02, and 5×10⁻⁴, respectively), whereas MAPK14 did not.

### 2.4.3. Validation of Survival Significance of RCC

To validate the survival significance of aging-related genes in independent dataset RCC, an analysis was performed with an independent dataset called Renal Cell Cancer-EU/FR (RECA-EU) of the International Cancer Genome Consortium. RECA-EU provided the gene expression data of normal RCC in which 17 of 45 RCC patients died. The mean age of the patients was 61 with a standard deviation of 10. In contrast to TCGA-KIRC, the survival status of the subjects in the RECA-EU dataset was not related with age (p-value 0.57 in the univariate Cox model).

Among 162 up-regulated aging-related genes and 87 down-regulated aging-related genes in TCGA-KIRC, RECA-EU had probes corresponding to 160 up-regulated genes and 85 down-regulated genes. Although the aging-related genes in TCGA-KIRC had no correlations with age in RECA-EU, the results of the univariate Cox model showed that the increasing index in RECA-EU had a hazard ratio of 1.60 and a p-value of 0.036, which were consistent with the TCGA-KIRC results.

The Kaplan-Meier model showed a p-value of 0.04 in the log-rank test with reference to the increasing index. However, the down-regulated aging-related identified in TCGA-KIRC had no prognostic power in RECA-EU (p-value 0.08 according to Cox model).

Finally, a Cox regression test was performed by using five up-regulated aging-related genes identified in the TCGA-KIRC data (DUSP22, MAPK14, MAPKAPK3, STAT1 and VCP) in the RECA-EU dataset to confirm the significance relationships between their expression and the patient survival (p-value 0.02 and hazard ratio 1.7).

2.4.4 Biological Roles of Aging-Related miRNAs in Kidneys

A literature search of the biological roles of aging-related miRNAs was performed. Bai et al. observed that overexpression of miR-335 and miR-34a induces premature aging of young hepatocytes in the kidneys through the inhibition of mitochondrial antioxidant enzymes SOD2 and TXNRD2 and simultaneously increases reactive oxygen species levels. Chen et al. demonstrated that down-regulation of miR-136-5p promotes cell proliferation, migration and invasion, whereas it inhibits apoptosis in RCC.

### 2.4.5 Deferentially Expressed Genes (DEG) in Renal Cancer

DESeq2 tool (RRID:SCR_000154) was used to identify a total of 423 up-regulated DEGs and 932 down-regulated DEGs in 72 KIRC patients. The up-regulated genes were enriched in the immune system and cytokine-related pathways, whereas the down-regulated genes were not significantly enriched in pathways.

The following 19 aging-related genes are DEGs in renal cancer: ABCG8, ADGRV1, CDH3, CGA, CPAMD8, CRISP2, DNER, ERP27, GABRP, LHFPL4, OR2I1P, PAPPA2, PCSK9, S100A2, SCEL, SLC16A5, STAP1, TMPRSS4 and UBD. In addition, the mean expression level of 423 up-regulated DEGs in the tumor tissues was correlated with the survival in the Cox model with a hazard ratio of 1.69 and a p-value of 0.01. However, the mean expression of 932 down-regulated DEGs in the tumor tissues corresponded to a hazard ratio of 0.87, thus indicating that there was no significant relationship with survival.

### 2.4.6 Survival Prediction Model

To compare the prognostic power of aging-related genes and cancer DEGs in KIRC, a survival prediction model was developed by using logistic regression and five input variables:

1. Mean expression of down-regulated cancer DEG in tumor tissues;

2. Mean expression of up-regulated cancer DEG in tumor tissues;

3. Decreasing index of normal tissues;

4. Increasing index of normal tissues; and

5. Combination of 2 and 4

In all cases, survival status was used as a dependent variable. Because the number of samples in KIRC (N=72) was not sufficient to generate consistent performance scores due to randomness in the division of the training set and test set, a 5-fold cross-validation was performed 1000 times for each model. The mean area under the ROC curve (AUC) of the model using the single input variable types from 1 to 4 was 0.483, 0.633, 0.703, and 0.748, respectively, suggesting that the expression value of aging-related genes is a better predictor of survival than cancer DEG. The increasing index in normal tissues showed the best predictive performance.

On the other hand, the mean expression of down-regulated cancer DEG showed the worst performance, because there was no significant relationship with survival according to the Cox model. In addition, the prediction model constructed by using the combined mean expression of up-regulated cancer DEG in tumor tissues and the increasing index in normal tissues as input variables showed an average AUC of 0.770, which was higher than any single input variable model. These results suggested that combining the information from cancer and normal tissues was more effective in predicting survival than using them individually. A boxplot of the calculated AUC is shown in FIG. 2D.

In order to avoid a decrease of the significance level of the correlation variables, known as multicollinearity, the increase and decrease indices were not used simultaneously due to the significant correlation.

### 2.5 Experimental Validation

### 2.5.1 Up-Regulated Expression of DUSP22, MAPK14, MAPKAPK3, STAT1 and VCP Genes in BMDM of Old Mice

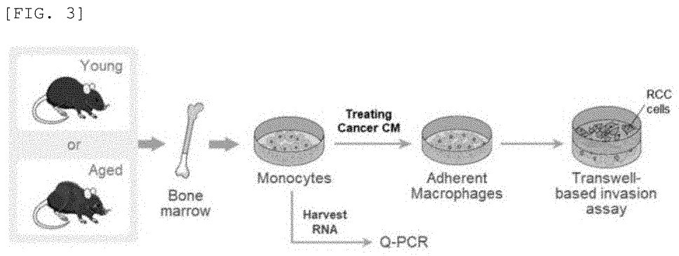

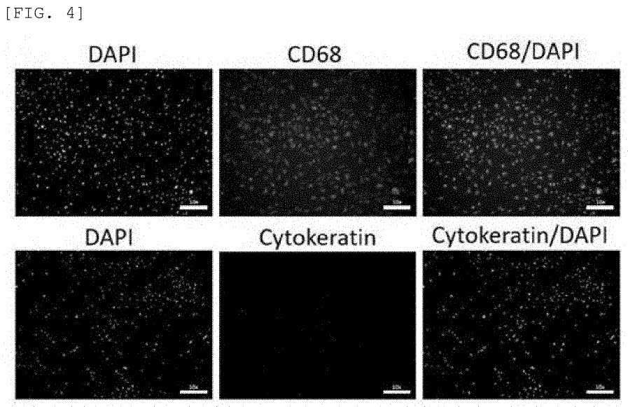

To validate the transcriptome analysis, a PCR-based gene expression analysis was performed on BMDMs isolated from young mice (5 weeks) or old (72 weeks) mice. BMDM was analyzed for the following reasons: (1) BMDM is derived from normal tissues; (2) BMDM is a tumor-associated macrophage (TAM) that is abundantly present at tumor sites, thus contributing to the immune response; and (3) BMDM is commonly used for human genetic biomarker analysis. The BMDM from young mice or old mice was differentiated into macrophages by culturing performed by using a cancer-conditioned medium (FIG. 3). The macrophage differentiation was confirmed by immunostaining for the marker CD68 (FIG. 4) and qPCR for F4/80, CD68, CD206 and iNOS. The secretion of macrophage differentiation factors by RCC cells was confirmed by using ELISA. The expression levels of DUSP22, MAPK14, MAPKAPK3, STAT1 and VCP genes were compared by qPCR.

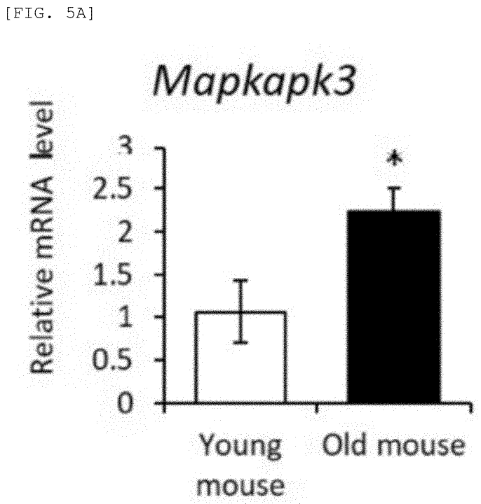

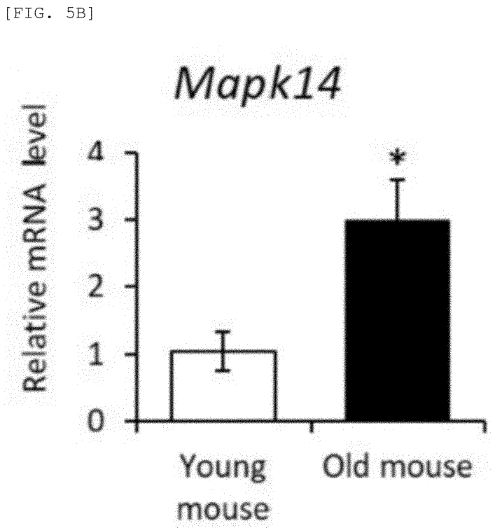

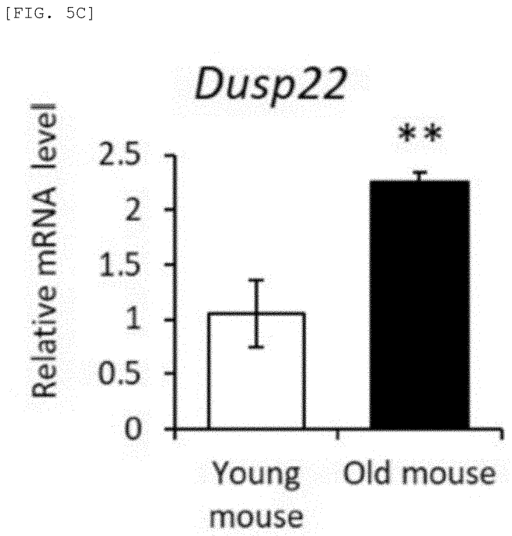

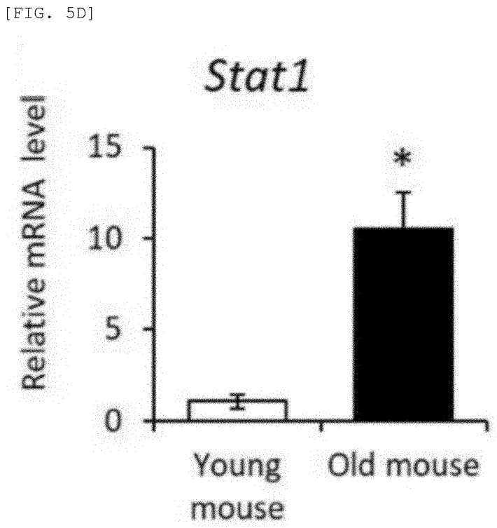

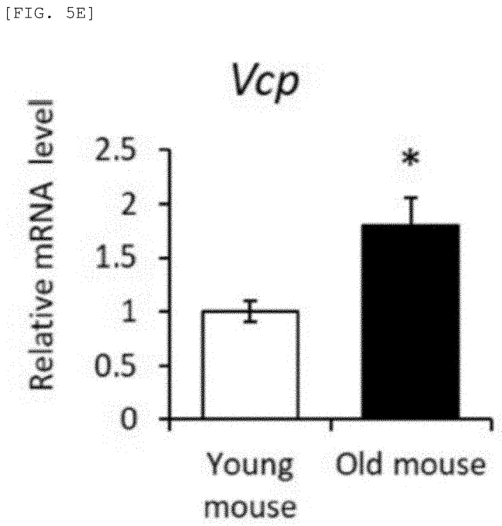

All aging-related genes identified by the transcriptomic analysis showed a more than 2-fold increase in the expression in the macrophages derived from the BMDM of the old mice, compared to the expression in the macrophages derived from the BMDM of young mice (FIGS. 5a-5e).

3.5.2. Inhibition of RCC Invasion by DUSP22 Knockdown of Macrophages Derived from Old Mice

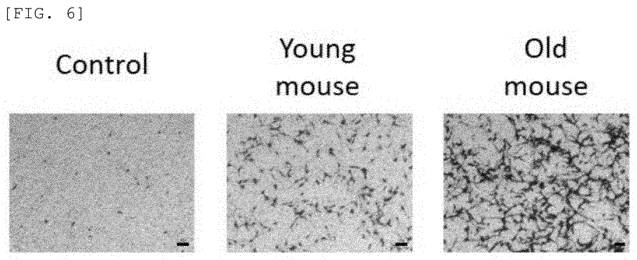

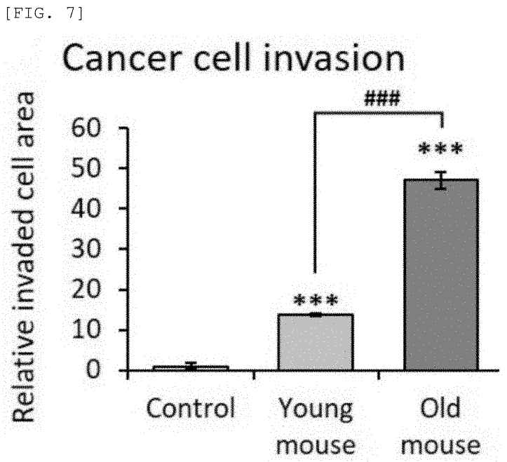

The survival rate of the KIRC cancer patients was correlated with the degree of metastasis of TCGA-KIRC, as determined by Fisher's exact test (p-value 4.4×10⁻⁵). Therefore, to confirm the effect of aging on metastasis, a cancer cell invasion analysis was performed by using RCC cells. The BMDM of old mice induced a more than 3-fold increase in RCC invasion, compared to the BMDM of young mice (FIGS. 6 and 7).

Among the five aging-related genes found in the TCGA-KIRC analysis, the expression level of DUSP22 was significantly related with metastasis. When the expression level of DUSP22 was analyzed by a t-test with the two groups of KIRC cancer patients regardless of the presence of metastasis, the p-value was 0.04. These results can be explained by the observation that DUSP22 induced the activation of c-Jun N-terminal kinase (JNK) through the apoptosis signal-regulating kinase 1-MAPK kinase 7-JNK1/2 axis. Activated JNK increases the secretion of epidermal growth factor (EGF) or stromal cell-derived factor 1 (SDF-1/CXCL12) to increase invasion and metastasis of tumor cells.

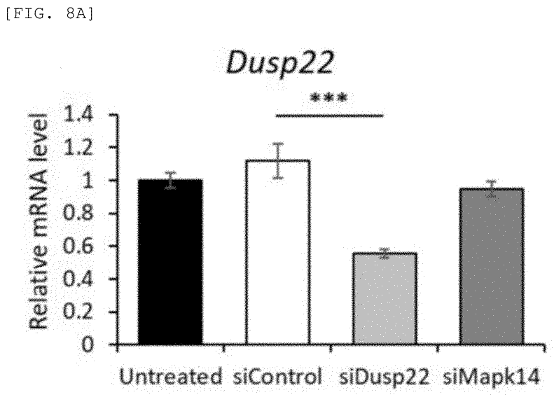

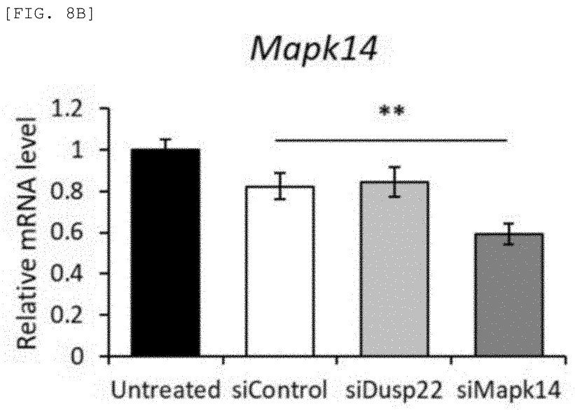

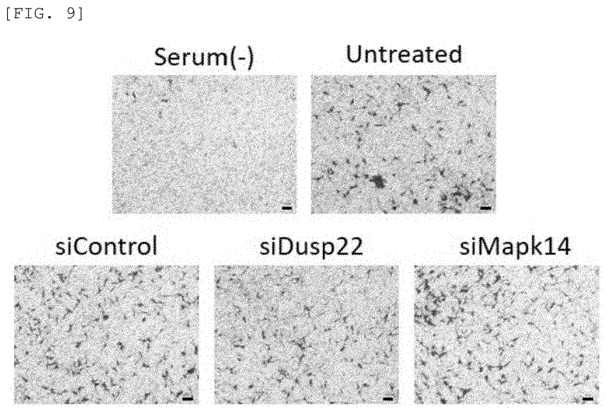

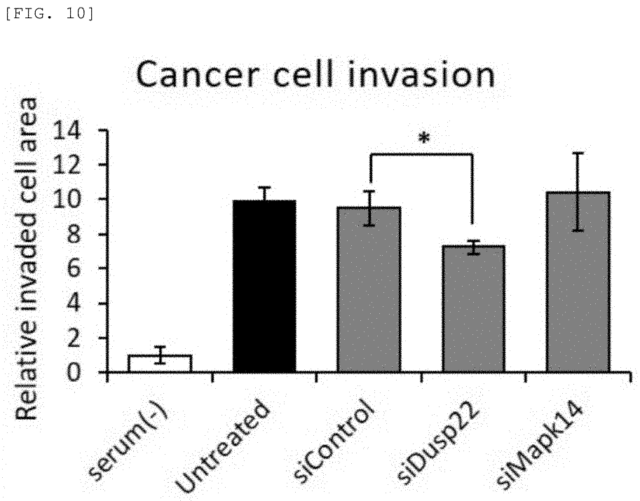

To evaluate the effect of DUSP22 on RCC cell invasion, DUSP22 knockdown was performed by siRNA. The mouse macrophage cell line RAW264.7 was used instead of BMDM due to the cytotoxicity generated by the transfection reagent. The knockdown of MAPK14, selected as a negative control, did not affect the invasion. On the contrary, the knockdown of DUSP22 significantly inhibited RCC cell invasion (FIGS. 8-10).

### 2.5.3. Promotion of Macrophage-Induced RCC Metastasis by DUSP22 In Vivo

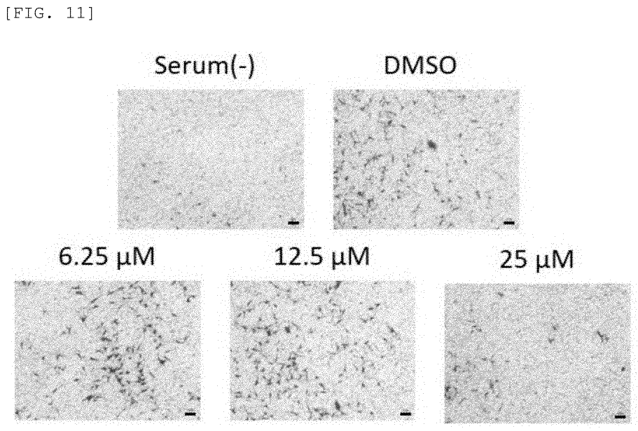

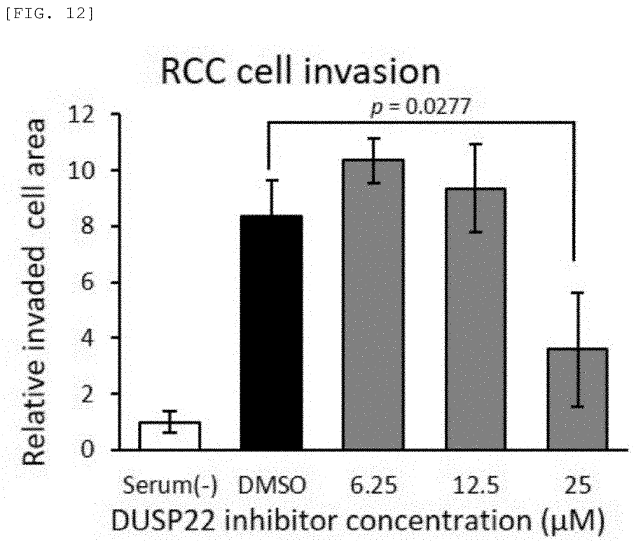

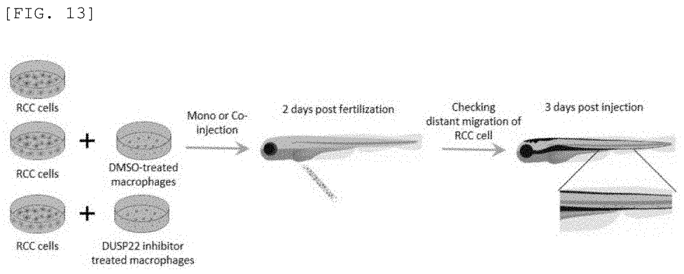

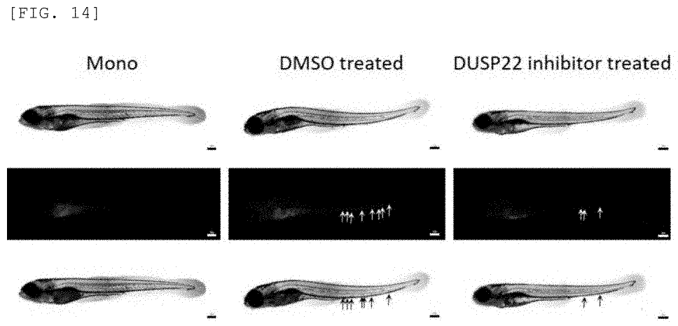

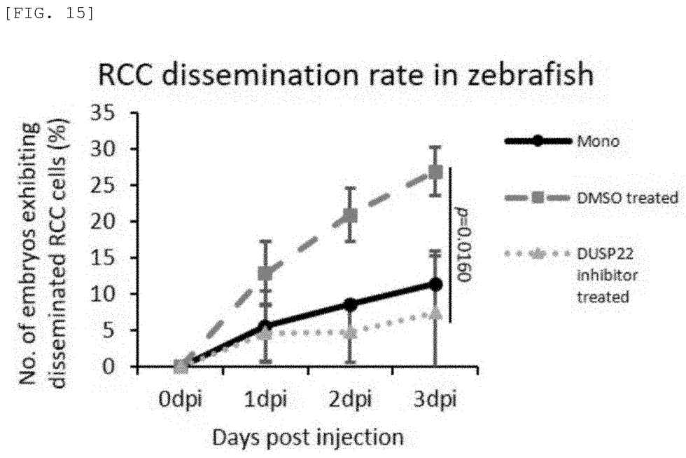

BML-260 is a small-molecule inhibitor of DUSP22. RCC invasion induced by the co-culture with macrophages was inhibited by BML-260 (FIGS. 11 and 12). A zebrafish cancer xenograft model was used to investigate the role of DUSP22 in RCC metastasis in vivo (FIG. 13). When the RCC cells were transplanted with macrophages, the rate of dissemination (early stage of metastasis) was higher than when only cancer cells were used. The treatment of macrophages with BML-260 before the co-transplantation with cancer cells significantly inhibited the dissemination in vivo (FIGS. 14 and 15).

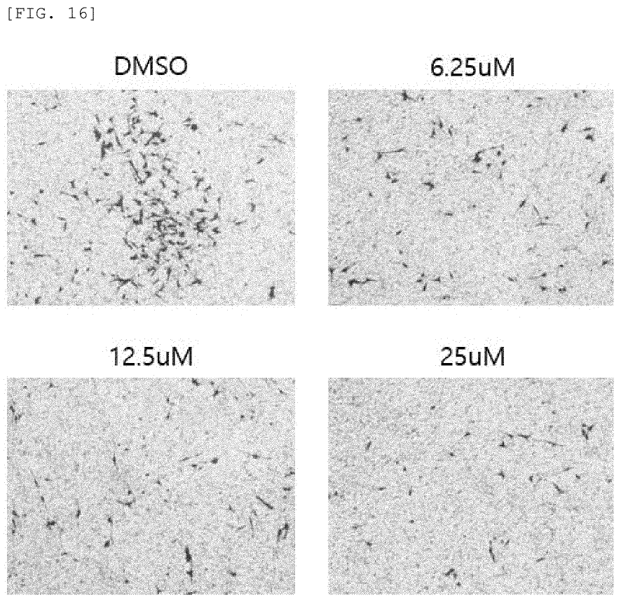

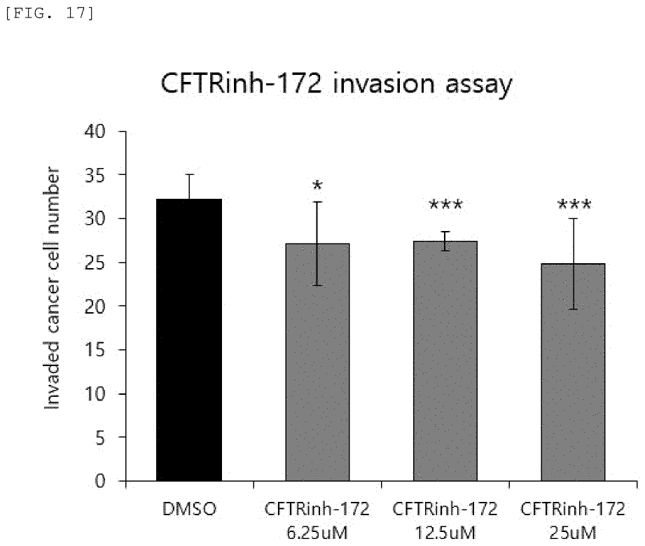

CFTRinh-172 is a derivative of BML-260. The RCC invasion induced by the co-culture with macrophages was inhibited by CFTRinh-172 (FIGS. 16 and 17).

### 3. Discussion

Aging of the population has increased both the incidence of cancer and its impact on society's health care costs. Therefore, there is a need to identify novel markers, disease progression regulators and new drug targets for various types of cancer. The inventors of the present invention present a novel transcriptome methodology to identify gene clusters having expression levels that correlate with aging in normal tissues adjacent to tumors that also correlate with patient survival. A protein-protein interaction network including these aging-related genes was constructed to find out functionally related gene clusters. As a result, many modules for various cancer types were identified. In addition to one module from KIRC mentioned in the section devoted to the results, 7, 1, 5 and 4 modules were found in BRCA, KICH, LUAD and THCA, respectively. The inventors of the present invention set up the minimum module size to be 5. For BRCA, which showed a greater number of aging-related genes than other tissue types, the minimum size was set to be 25.

In RCC, several mutated genes that are related with metabolic, immune, genomic and therapeutic-related external pressures have been identified as the key genes involved in development. In addition, tumor progression and expression levels of 16 genes have been suggested and validated as predictors of the recurrence of RCC. However, there have been no studies to investigate aging-related genes as prognostic markers of RCC. Among the genetic predictors of aging-related survival in KIRC, the inventors of the present invention identified DUSP22 as a metastasis-related gene that promotes RCC cell invasion. These results are the first proof that DUSP22 is a prognostic marker as well as a regulator of RCC progression.

The mechanism by which DUSP22 may affect RCC invasion can be deduced from previous publications showing that DUSP22 activates c-JNK through the apoptosis signal-regulated kinase 1-MAPK kinase 7-JNK1/2 axis. The activation of c-JNK increases the secretion of EGF and SDF-1/CXCL12, which are two enhancers of cancer cell migration. The knockdown of the DUSP22 gene inhibited the invasion of RENCA cells, which are used as a model for RCC. Therefore, DUSP22 may modulate the invasion in other forms of RCC, such as papillary RCC and chromogenic RCC.

The transcript analysis was performed with the samples obtained from genetically normal tissues adjacent to the tumor, and the prognostic markers of survival were validated in BMDM. The inventors of the present invention selected the cell model, because BMDMs invade tumor tissues, differentiate into TAMS that are adjacent to tumor cells, and are key regulators of cancer progression. In addition, a BMDM biopsy samples can be easily obtained from RCC patients for a genetic analysis. A previous study investigated aging-related variations in gene expression patterns in RCC. However, this study did not include a transcriptome analysis of patient survival and only the aging-related pathways were reported in RCC.

Although several new therapies were approved over the past decade, there still remains an urgent need to develop new drug targets for RCC and expand therapeutic equipment. There are no drugs that are reported in RCC to be targeted to a specific molecule. The present invention indicates that DUSP22 can be an attractive target of a screening protocol for developing specific drugs for RCC.

RCC is the most fatal type of genitourinary tumor. Early diagnosis and prompt treatment will greatly improve the patient survival. Early diagnosis can also help to improve disease progression and avoid inadequate treatment. Sensitive prognostic biomarkers can facilitate early detection and progression monitoring. Previous studies have reported prognostic markers for RCC, such as B7-H1, carbonic anhydrase-IX and PTEN. More recent studies have focused on cell-based functions, such as aberrant alternative splicing features and DNA methylation markers.

In the present invention, a novel approach was investigated to discover prognostic markers based on aging-related genes expressed in normal tissues that are related with patient survival. The five genes found (DUSP22, MAPK14, MAPKAPK3, STAT1, VCP) have the potential to further improve the current biomarkers that have already been developed for RCC, and can be analyzed by using normal, easily assessed biopsy samples obtained from bone marrow-like tissues. In addition, these markers may be subject to subsequent investigation to further characterize the roles of immune cells and aging in RCC progression.

### 4. Conclusions

The transcriptome analysis of the present invention showed that aging-related gene expression in normal tissues can predict the survival of cancer patients. The inventors of the present invention identified five prognostic markers of RCC, expressed as DUSP22, MAPK14, MAPKAPK3, STAT1 and VCP, as well as DUSP22 as a RCC regulation factor and a new target of RCC metastasis. These marker genes can improve the biomarker set that is currently available for RCC. Due to the considerable proportion of patients with RCC diagnosed as a metastatic disease, these results can potentially facilitate diagnosis and treatment of renal cancer. DUSP22 can be an attractive candidate for further development as a specific molecular drug target for RCC. Moreover, this novel approach to transcriptomics can be applied to identify additional sets of prognostic markers for various cancer types, as future patient survival data become available.

The description of the present invention above is for illustration, and those of ordinary skill in the art to which the present invention pertains can understand that it can be easily modified into other specific forms without changing the technical principles or essential features of the present invention. Therefore, it should be construed that the embodiments described above are illustrative in all respects and not restrictive. For example, each component described as a single type may be implemented in a dispersed form, and likewise, components described as distributed may be implemented in a combined form.

The scope of the present invention is indicated by the claims described below, and all changes or modifications derived from the meaning and scope of the claims and their equivalents should be construed as being included in the scope of the present invention.

What is claimed is:

1. A method for screening markers for predicting prognosis of renal cell carcinoma (RCC) using transcriptomic analysis, comprising:
a) identifying a group of aging-related genes by integrating the gene expression data and protein-protein interaction data of a normal tissue of RCC patients;
b) analyzing relationship between expression of aging-related genes in the normal tissue and survival of cancer patients; and
c) determining an aging-related gene that is up-regulated in the normal tissue and shows a significant relationship with survival of RCC patients as a marker for predicting RCC prognosis,
to provide necessary information about RCC prognosis.

2. The method for screening markers for predicting prognosis of RCC using transcriptomic analysis according to claim 1, wherein the a) is performed by using a linear regression model.

3. The method for screening markers for predicting prognosis of RCC using transcriptomic analysis according to claim 1, wherein the b) comprises validating the effect of aging-related gene expression on cancer cell invasion and metastasis through in vitro and in vivo experiments using aging animal models.

4. The method for screening markers for predicting prognosis of RCC using transcriptomic analysis according to claim 1, wherein the aging-related gene determined as a marker for predicting RCC prognosis in c) is at least one gene selected from the group consisting of DUSP22, MAPK14, MAPKAPK3, STAT1, and VCP.

5. The method for screening markers for predicting prognosis of RCC using transcriptomic analysis according to claim 1, wherein the RCC is clear cell renal cell cancer.

6. A marker composition for predicting renal cell carcinoma (RCC) prognosis, comprising at least one below
(a) at least one gene selected from the group consisting of DUSP22, MAPK14, MAPKAPK3, STAT1, and VCP; and
(b) protein encoded by at least one gene selected from the group consisting of DUSP22, MAPK14, MAPKAPK3, STAT1, and VCP.

7. A marker composition for predicting RCC prognosis according to claim 6, wherein the RCC is clear cell renal cell cancer.

8. A method of treating renal cell carcinoma (RCC), comprising administering a pharmaceutical composition comprising a DUSP22 inhibitor as an active ingredient to a subject.

9. The method of claim 8, wherein the DUSP22 inhibitor inhibits invasion and metastasis of RCC.

10. The method of claim 8, wherein the DUSP22 inhibitor is a compound represented by Formula 1 below.

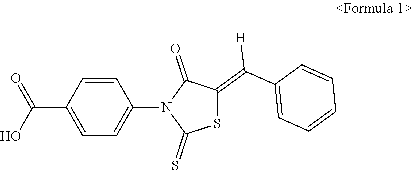 [SMILES: O=C(O)c1ccc(N2C(=O)/C(=C/c3ccccc3)SC2=S)cc1]

11. The method of claim 8, wherein the DUSP22 inhibitor is a compound represented by the Formula below.

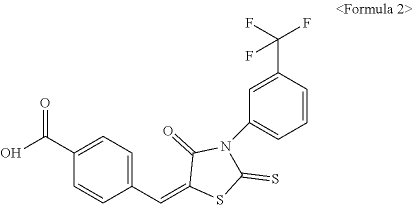 [SMILES: O=C(O)c1ccc(/C=C2/SC(=S)N(c3cccc(C(F)(F)F)c3)C2=O)cc1]

12. The method of claim 8, wherein the RCC is clear cell renal cell cancer.
